(19) United States
(12) Patent Application Publication
(10) Pub. No.: US 2026/0357316 A1
(43) Pub. Date: Nov. 12, 2026

(54) METHODS AND SYSTEMS FOR REGENERATIVE SYNTHESIS OF OLIGONUCLEOTIDES

(71) Applicant: Atlas Data Storage, Inc., South San Francisco, CA (US)

(72) Inventor: Manasi Madhav RAJE, South San Francisco, CA (US)

(21) Appl. No.: 19/662,812

(22) Filed: Apr. 29, 2026

Related U.S. Application Data
(60) Provisional application No. 63/797,523, filed on Apr. 30, 2025.

(51) Int. Cl. C12P 19/34; C12N 9/22; C12N 9/24; C12N 9/88

(52) U.S. Cl. CPC C12P 19/34; C12N 9/22; C12N 9/2497; C12N 9/88; C12Y 301/03036; C12Y 301/21002; C12Y 301/21007; C12Y 302/02027; C12Y 402/99018

(57) ABSTRACT

Described herein are methods and systems for enzymatic synthesis of oligonucleotides, such as DNA, on a functionalized substrate. Further described herein are methods and systems for reusing a substrate already used for enzymatic synthesis of oligonucleotides, without the need for re-functionalization. The described methods and systems reduce or eliminate interruptions to the synthesis process, allowing for efficient and rapid oligonucleotide synthesis.

### CROSS-REFERENCE TO RELATED APPLICATIONS

This application claims priority to and the benefit of U.S. Provisional Application No. 63/797,523, filed Apr. 30, 2025, and titled “Surfaces for Enzymatic DNA Synthesis and Methods Thereof,” the disclosure of which is hereby incorporated by reference herein in its entirety.

### SEQUENCE LISTING

The Sequence Listing XML associated with this application is provided electronically in XML format and is hereby incorporated by reference into the specification. The name of the XML file containing the Sequence Listing XML is “355893-2387_seqlist.xml”. The XML file is 30,840 bytes, created on Apr. 29, 2026, and is being submitted electronically via USPTO Patent Center.

### BACKGROUND

Methods of producing synthetic polymers (e.g., oligonucleotide molecules, such as DNA molecules), may include using a functionalized surface containing a template oligonucleotide. However, after extracting the synthetic oligonucleotide molecules, the functionalized surface cannot generally be reused for another round of synthesis. Re-functionalization generally includes removal of the surface from the synthesizer, exposure to chemically harsh conditions that weaken the surface itself, and re-insertion into the synthesizer. Further, these processes impose additional costs, time, and opportunities to introduce error or contaminants into the synthesis process.

### SUMMARY

Embodiments described herein relate to methods and systems for enzymatic synthesis of oligonucleotides, such as DNA, on a functionalized substrate. Further described herein are methods and systems for reusing a substrate already used for enzymatic synthesis of oligonucleotides, without the need for re-functionalization. The described methods and systems reduce or eliminate interruptions to the synthesis process, allowing for efficient and rapid oligonucleotide synthesis.

In one aspect, disclosed herein is a method for oligonucleotide synthesis, the method comprising providing a substrate including an oligonucleotide, the oligonucleotide including a first end bound to the substrate and a second end opposite the first end; adding a cleavable base to the second end of the oligonucleotide; enzymatically extending the oligonucleotide to generate a DNA molecule; and cleaving the DNA molecule at the cleavable base. In some embodiments, the DNA molecule is a first DNA molecule and the cleavable base is a first cleavable base, and the method further comprises: adding a second cleavable base to the second end of the oligonucleotide; enzymatically extending the oligonucleotide to generate a second DNA molecule; and cleaving the second DNA molecule at the second cleavable base. In some embodiments, cleaving the DNA molecule at the cleavable base generates a 3′-phosphate group on the second end of the oligonucleotide, and the method further comprises enzymatically removing the 3′-phosphate group from the second end of the oligonucleotide.

In some embodiments, the substrate comprises a silicon surface, a silicon dioxide surface, a silicon nitride surface, a platinum surface, a gold surface, an indium tin oxide surface, a conductive surface, a non-conductive surface, or a composite surface. In some embodiments, the surface is functionalized to enable binding of the oligonucleotide. In some embodiments, the surface is functionalized with a silane coupling agent.

In some embodiments, the method further comprises forming the oligonucleotide on the substrate via phosphoramidite chemistry.

In some embodiments, adding the cleavable base to the second end of the oligonucleotide comprises contacting the substrate with deoxyuridine triphosphate, terminal deoxynucleotidyl transferase, a coupling buffer, or a combination thereof. In some embodiments, cleaving the DNA molecule comprises contacting the substrate to an enzyme capable of cleaving at the cleavable base. In some embodiments, the cleavable base is a uracil base, and the enzyme comprises uracil DNA glycosylase. In some embodiments, the cleavable base is a uracil base, and cleaving the DNA molecule further comprises contacting the substrate to a compound or enzyme capable of breaking a phosphodiester bond at an abasic site. In some embodiments, the cleavable base is a uracil base, and the enzyme further comprises Endonuclease VIII or Endonuclease IV. In some embodiments, the cleavable base is an inosine base, and the enzyme comprises Endonuclease V.

In some embodiments, enzymatically removing the 3′-phosphate group from the second end of the oligonucleotide comprises exposing the substrate to a 5′-phosphatase enzyme. In some embodiments, the 5′-phosphatase enzyme comprises T4 polynucleotide kinase, Antarctic phosphatase, calf intestinal alkaline phosphatase, or a combination thereof.

In another aspect, disclosed herein is a method for oligonucleotide synthesis, the method comprising providing an oligonucleotide comprising a first end and a second end; adding a cleavable base to the second end of the oligonucleotide; contacting the oligonucleotide to a substrate, wherein the contacting binds the first end of the oligonucleotide to the substrate; enzymatically extending the oligonucleotide to generate a DNA molecule; cleaving the DNA molecule at the cleavable base, thereby generating a 3′-phosphate group on the second end of the oligonucleotide; and enzymatically removing the 3′-phosphate group from the second end of the oligonucleotide. In some embodiments, the DNA molecule is a first DNA molecule and the cleavable base is a first cleavable base, and the method further comprises adding a second cleavable base to the second end of the bound oligonucleotide; enzymatically extending the oligonucleotide to generate a second DNA molecule; and cleaving the second DNA molecule at the second cleavable base.

In some embodiments, cleaving the DNA molecule at the cleavable base generates a 3′-phosphate group on the second end of the oligonucleotide, and the method further comprises enzymatically removing the 3′-phosphate group from the second end of the oligonucleotide.

In some embodiments, the substrate comprises a silicon surface, a silicon dioxide surface, a silicon nitride surface, a platinum surface, a gold surface, an indium tin oxide surface, a conductive surface, a non-conductive surface, or a composite surface. In some embodiments, the surface is functionalized to enable binding of the oligonucleotide. In some embodiments, the surface is functionalized with a silane coupling agent.

In some embodiments, the method further comprises forming the oligonucleotide on the substrate via phosphoramidite chemistry.

In some embodiments, adding the cleavable base to the second end of the oligonucleotide comprises contacting the substrate with at least one of deoxyuridine triphosphate, terminal deoxynucleotidyl transferase, or a coupling buffer. In some embodiments, cleaving the DNA molecule comprises contacting the substrate to an enzyme capable of cleaving at the cleavable base. In some embodiments, the cleavable base is a uracil nucleotide, and the enzyme comprises uracil DNA glycosylase. In some embodiments, the cleavable base is a uracil nucleotide, and cleaving the DNA molecule further comprises contacting the substrate to a compound or enzyme capable of breaking a phosphodiester bond at an abasic site. In some embodiments, the cleavable base is a uracil nucleotide, and the enzyme further comprises Endonuclease VIII or Endonuclease IV. In some embodiments, the cleavable base is an inosine nucleoside, and the enzyme comprises Endonuclease V.

In some embodiments, enzymatically removing the 3′-phosphate group from the second end of the oligonucleotide comprises exposing the substrate to a 5′-phosphatase enzyme. In some embodiments, the 5′-phosphatase enzyme comprises T4 polynucleotide kinase, Antarctic phosphatase, calf intestinal alkaline phosphatase, or a combination thereof.

In another aspect, disclosed herein is a system for oligonucleotide synthesis, the system comprising a substrate comprising a surface and an oligonucleotide bound to the surface, wherein the oligonucleotide comprises a first end bound to the surface and a second end comprising a cleavable base; a first enzyme configured to extend the oligonucleotide at the cleavable base; and a second enzyme configured to cleave the oligonucleotide at the cleavable base, thereby generating a 3′-phosphate group on the second end of the oligonucleotide. In some embodiments, the second enzyme is configured to generate a 3′-phosphate group on the second end of the oligonucleotide, and the system further comprises a third enzyme configured to remove the 3′-phosphate group from the second end of the oligonucleotide. In some embodiments, the third enzyme is a 5′-phosphatase enzyme. In some embodiments, the 5′-phosphatase enzyme comprises at least one of T4 polynucleotide kinase, Antarctic phosphatase, or calf intestinal alkaline phosphatase.

In some embodiments, the surface is a silicon surface, a silicon dioxide surface, a silicon nitride surface, a platinum surface, a gold surface, an indium tin oxide surface, a conductive surface, a non-conductive surface, or a composite surface. In some embodiments, the surface is functionalized to enable binding of the oligonucleotide. In some embodiments, the surface is functionalized with a silane coupling agent.

In some embodiments, the cleavable base is a uracil base and the second enzyme comprises uracil DNA glycosylase. In some embodiments, the cleavable base is a uracil base and the second enzyme further comprises Endonuclease VIII or Endonuclease IV. In some embodiments, the cleavable base is an inosine base and the second enzyme comprises Endonuclease V.

### BRIEF DESCRIPTION OF THE DRAWINGS

Embodiments of the present disclosure may be implemented in connection with aspects illustrated in the attached drawings. These drawings show different aspects of the present disclosure, and where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. For simplicity and clarity of illustration, the figures depict the general structure and/or manner of construction of the various embodiments/aspects described herein.

FIGS. 1A-1C are flowcharts for an exemplary method for synthesizing an oligonucleotide on a surface and regenerating the surface to continue oligonucleotide synthesis, according to aspects of the present disclosure.

FIG. 2 is a schematic illustration of an exemplary functionalized surface, according to aspects of the present disclosure.

FIG. 3 is a flowchart for an exemplary method for synthesizing an oligonucleotide on a surface and regenerating the surface to continue oligonucleotide synthesis, according to aspects of the present disclosure.

FIGS. 4A-4C are chromatograms including LC-MS results from an exemplary method for cleaving an exemplary synthetic oligonucleotide in solution, according to aspects of the present disclosure.

FIGS. 5A-5C are chromatograms including LC-MS results from an exemplary method for cleaving an exemplary synthetic oligonucleotide in solution, according to aspects of the present disclosure.

FIGS. 6A-6B are graphs depicting mass spectrometry results from an exemplary method for binding an exemplary synthetic oligonucleotide to a surface, according to aspects of the present disclosure.

FIG. 7 is a graph depicting ultraviolet-visible (UV-vis) results from an exemplary method for synthesizing an oligonucleotide on a surface and regenerating the surface to continue oligonucleotide synthesis, according to aspects of the present disclosure.

FIGS. 8A-8D are chromatograms including LC-MS results from an exemplary method for synthesizing, cleaving, repairing, and reusing an oligonucleotide in solution, according to aspects of the present disclosure.

FIG. 9 is a schematic illustration of an exemplary method for synthesizing an oligonucleotide on a surface and reusing the surface to continue oligonucleotide synthesis, according to aspects of the present disclosure.

FIG. 10 is a chromatogram including LC-MS results from an exemplary method for cleaving an exemplary oligonucleotide on a surface, according to aspects of the present disclosure.

FIG. 11 is a chromatogram including LC-MS results from an exemplary method for synthesizing an oligonucleotide on a surface and reusing the surface to continue oligonucleotide synthesis, according to aspects of the present disclosure.

### DETAILED DESCRIPTION

The methods described herein may be carried out in an aqueous buffer environment. This allows for integration of the disclosed methods into systems used for DNA synthesis. The integration allows for a cyclic reuse of functionalized surfaces within the platform used for the synthesis itself. Further, the aqueous waste product of the disclosed methods may have a lower environmental impact than traditional methods, and may result in decreased hazardous waste disposal costs.

As used herein, the term “about,” or “approximately” is meant to account for variations, for example, due to experimental error. When applied to numeric values, the terms “about” and “approximately” may indicate a variation of +/−5% from the disclosed numeric value, unless a different variation is specified. As used herein, the singular forms “a,” “an,” and “the” include plural referents unless the context clearly dictates otherwise. Further, all ranges are understood to be inclusive of endpoints, e.g., from 1 nanometer (nm) to 5 nm would include lengths of 1 nm, 5 nm, and all distances between 1 nm and 5 nm.

As used herein, the terms “oligonucleotide” or “polynucleotide” refer to a nucleic acid molecule, such as deoxyribonucleic acid (DNA) or ribonucleic acid (RNA). A nucleic acid molecule is made up of one or more nucleic acid bases, such as one or more nucleotides (e.g., uracil) or one or more nucleosides (e.g., inosine), connected via phosphate linkages or sulfur-containing linkages. A nucleic acid molecule may include a 5′ end and a 3′ end. As used herein, the term “downstream” may refer to a direction along the nucleic acid molecule moving towards the 3′ end, and the term “upstream” may refer to a direction along the nucleic acid molecule moving towards the 5′ end. As used herein, the term “cleavable base” may refer to a base capable of being cleaved, unbound, or unattached from another base, molecule, or surface. As used herein, the term “synthetic oligonucleotide” refers to an oligonucleotide created through directed oligonucleotide synthesis, such as the methods described below, rather than by extraction from a natural compound.

As used herein, the terms “enzymatic oligonucleotide synthesis” or “enzymatic DNA synthesis” refer to methods of synthesizing oligonucleotides through the use of enzymes. “Enzymatic removal” or “enzymatic cleavage” refers to the extraction or cleavage of a synthetic oligonucleotide from another molecule or surface.

As used herein, the term “electrochemical chip” or “chip” refer to a substrate used for oligonucleotide synthesis. Such chips may include one or more surfaces, which may be functionalized for association with a base, sugar, and/or phosphate of an oligonucleotide.

As used herein, the terms “ligate” or “ligation” refer to the binding or joining of two oligonucleotides by a phosphate ester linkage. For example, two single strands of DNA may be ligated to form a double-stranded DNA. Ligation may be carried out by an enzyme, such as DNA ligase, which catalyzes formation of the phosphate ester linkage.

As used herein, the term “solubility tag” refers to a small molecule or molecular group or moiety attached to an oligonucleotide to enhance solubility of the oligonucleotide in a solution. Examples of solubility tags for oligonucleotides include ionic liquids and polyethylene glycol (PEG).

### Methods for Regenerative DNA Synthesis

Provided herein are methods for producing synthetic oligonucleotides, such as synthetic DNA molecules. As detailed further below, methods described herein enable a regenerative synthesis process in which a functionalization of the surface is generated and regenerated without damaging the underlying surface. Further, all or some of the operations described herein may be performed in an aqueous buffer environment, producing aqueous waste products that may easily and safely disposed of.

Generally, the methods described herein may involve reuse and rework of a surface for enzymatic synthesis. As used herein, reuse refers to the process of using the same synthesis sites after cleavage of the previously synthesized material. Rework refers to the process of re-establishing the original template strand used for the first synthesis. The methods described herein provide for both reuse and rework of a surface for enzymatic synthesis. For example, the methods provided herein may generally include: establishing an attachment layer and template strand on the surface; incorporating a cleavable layer; performing enzymatic oligonucleotide synthesis; cleaving and extracting the synthesized material. At this point, the methods described herein will allow for either reuse of the surface to incorporate another cleavable layer, or rework of the surface to again establish an attachment layer and template strand.

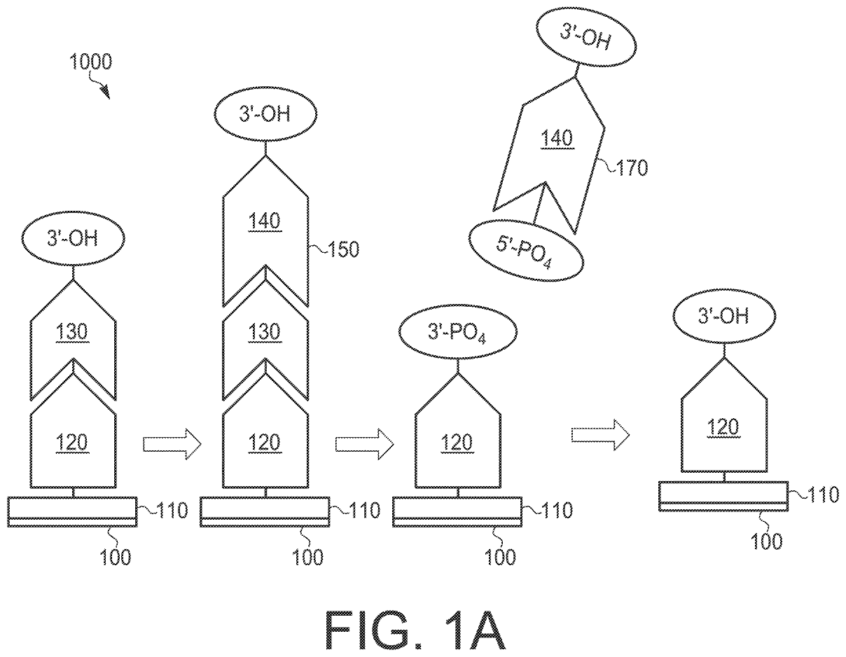

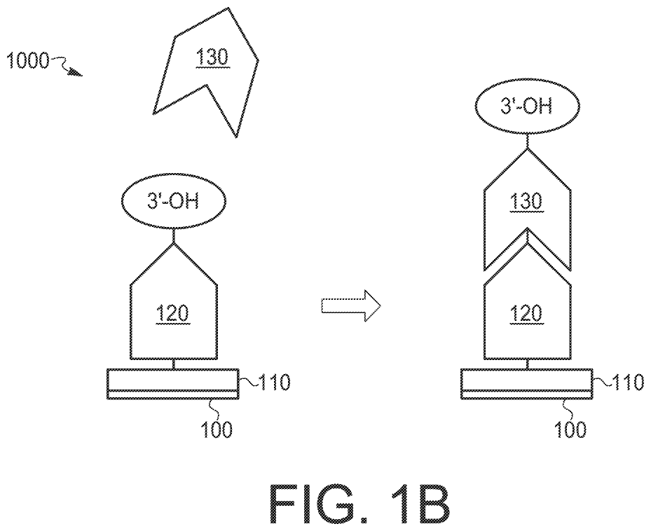

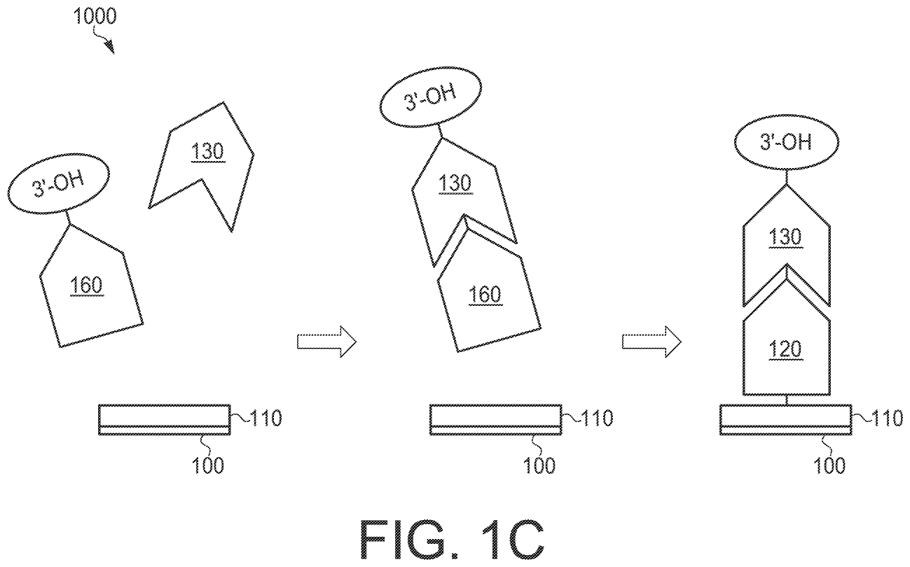

A flowchart describing an exemplary method 1000 for synthesizing and cleaving an oligonucleotide on a substrate 100, and regenerating the surface 110 for continued synthesis is shown in FIG. 1A. A substrate 100 including a functionalized surface 110 may be prepared with an oligonucleotide 120 bound to the surface 110. For example, a 5′ end of the oligonucleotide 120 may be bound to surface 110. The bound oligonucleotide 120 may include a 3′-OH hydroxyl group at a 3′ end. The surface-bound oligonucleotide 120 may be any sequence of one or more nucleic acid bases. In some embodiments, the surface-bound oligonucleotide 120 may include a minimum number of nucleic acid bases needed for extension by an enzyme, as described below. The surface-bound oligonucleotide 120 may be a polymer of one type of nucleic acid base (e.g., two or more thymine bases, two or more adenine bases, two or more cytosine bases, or two or more guanine bases) or a combination of nucleic acid bases. In some embodiments, the surface-bound oligonucleotide 120 may include a nucleic acid base sequence that is a preferred motif for a starting oligonucleotide and/or a nucleic acid base sequence that is intended for use in one or more downstream processes. The surface-bound oligonucleotide 120 may include at least one thymine base. In some embodiments, the surface-bound oligonucleotide 120 may include more than one (e.g., 2, 3, 4, 5, 6, 7, 8, 9, 10, etc.) thymine bases. The surface-bound oligonucleotide 120 may include modified nucleic acid bases, dye-containing nucleic acid bases, spacers (e.g., polyethylene glycol), and other modifications.

The surface-bound oligonucleotide 120 may be extended with the addition of at least one cleavable base 130 to the surface-bound oligonucleotide 120. For example, as shown in FIG. 1B, the at least one cleavable base 130 may be added to a 3′ of the surface-bound oligonucleotide 120. The at least one cleavable base 130 may be enzymatically added to the surface-bound oligonucleotide 120. The at least one cleavable base 130 may include a nucleotide and/or nucleoside. Adding the at least one cleavable base 130 may include contacting the surface-bound oligonucleotide 120 with one or more enzymes. The one or more enzymes may include terminal deoxynucleotidyl transferase (TdT), cytosine deaminase, variants of TdT, or a combination thereof. TdT or TdT variants may be provided with or without solubility tags.

As used herein, the terms “uracil DNA glycosylase”, “UDG”, and “UDG enzyme” may refer to an enzyme that nonspecifically binds to a polynucleotide and catalyzes excision of a uracil base from the phosphodiester backbone of the polynucleotide, creating an abasic site where the uracil base once was located. Creation of the abasic site may initiate and/or facilitate breaking of the phosphodiester bond at the abasic site by another enzyme or chemical, such as Endonuclease VIII, endonuclease IV, or an AMA mixture containing a 1:1 (v/v) solution of 30% ammonium hydroxide and 40% aqueous methylamine, separating the polynucleotide at the abasic site into two fragments.

For example, the at least one cleavable base 130 may include one uracil base. In some embodiments, the at least one cleavable base 130 includes more than one (e.g., 2, 3, 4, 5, 6, 7, 8, 9, 10, etc.) uracil bases. One or more bases of the at least one cleavable base 130 may be protected. In some embodiments, one or more bases of the at least one cleavable base 130 may be unprotected.

In some embodiments, the at least one cleavable base 130 may be a nucleoside, and may be chemically or enzymatically added to the 3′ end of the surface-bound oligonucleotide 120. For example, the at least one cleavable base 130 may be at least one inosine base. The at least one inosine base may be added to the surface-bound oligonucleotide by an enzyme. For example, the surface-bound oligonucleotide may be contacted with a mixture containing one or more enzymes, such as TdT enzyme or a variant thereof. The at least one inosine base may be added to the surface-bound oligonucleotide by a chemical reaction, such as phosphoramidite chemistry. For example, the surface-bound oligonucleotide may be contacted with inosine phosphoramidite. In some embodiments, more than one (e.g., 2, 3, 4, 5, 6, 7, 8, 9, 10, etc.) inosine bases may be added to the surface-bound oligonucleotide.

In some embodiments, the at least one cleavable base 130 may be a combination of nucleic acid bases, e.g., a combination of cleavable bases. In some embodiments, the at least one cleavable base 130 may be a combination of nucleic acid bases including a specific sequence motif that can be recognized by a cleavage enzyme.

In some embodiments, the at least one cleavable base 130 may be added to the surface-bound oligonucleotide 120 before the oligonucleotide 120 is bound to the surface 110. For example, as shown in FIG. 1C, the at least one cleavable base 130 may be added enzymatically to an oligonucleotide 160 in solution, by contacting the oligonucleotide 160 to a solution containing the at least one cleavable base 130 and one or more enzymes. The one or more enzymes may include TdT, cytosine deaminase, cytosine deamination, dUTP, variants of TdT, or a combination thereof. The oligonucleotide 160 may include at least one thymine base. In some embodiments, the oligonucleotide 160 may include more than one (e.g., 2, 3, 4, 5, 6, 7, 8, 9, 10, etc.) thymine bases. TdT or TdT variants may be provided with or without solubility tags. In some embodiments, one uracil base may be added to the oligonucleotide 160 in solution. In some embodiments, more than one (e.g., 2, 3, 4, 5, 6, 7, 8, 9, 10, etc.) uracil base may be added to the oligonucleotide 160 in solution. In some embodiments, the at least one uracil base may be protected or unprotected. The at least one uracil base may be added to the oligonucleotide 160 to form a uracil-containing oligonucleotide, which may then be incorporated (e.g., bound) at its 5′ end to the surface 110 to form a surface-bound oligonucleotide 120. The surface-bound oligonucleotide 120, bound at its 5′ end to the surface 110, may be attached at its 5′ end to the at least one uracil base.

Returning now to FIG. 1A, the surface-bound oligonucleotide 120 including the at least one cleavable base 130 may be extended to form product oligonucleotide 140, thereby generating a complex 150 including the product oligonucleotide 140, the at least one cleavable base 130, and the surface-bound oligonucleotide 120. For example, nucleic acid bases in the sequence of a desired synthetic product oligonucleotide 140 may be added to the 5′ end of the at least one cleavable base 130 attached to the surface-bound oligonucleotide 120. The product oligonucleotide 140 may be added to the at least one cleavable base 130 enzymatically, as described herein.

After the complex 150 is generated, product oligonucleotide 140 may be cleaved from surface-bound oligonucleotide 120. For example, complex 150 may be contacted by one or more enzymes that target the at least one cleavable base 130. The one or more enzymes may cleave complex 150 at the at least one cleavable base 130. Cleaving of the at least one cleavable base 130 may result in separation of a fragment 170 including the product oligonucleotide 140. In addition or alternatively, fragment 170 may include a phosphate group and/or a hydroxyl group. For example, a 3′ end of fragment 170 may include a hydroxyl group and/or a 5′ end of fragment 170 may include a phosphate group. The product oligonucleotide 140 may be positioned between a hydroxyl group at the 3′ end of fragment 160 and a phosphate group at a 5′ end of fragment 170. After product oligonucleotide 140 is cleaved from surface-bound oligonucleotide 120, surface-bound oligonucleotide 120 may include a phosphate group at a 3′ end.

As described herein, the at least one cleavable base 130 may be targeted for cleavage by one or more enzymes that specifically cleave a molecule at the site of the at least one cleavable base 130. Any known strand-cleaving enzyme may be used, including enzymes that have been engineered to improve the specific cleavage activity. For example, the at least one cleavable base 130 may include at least one uracil base, and may be cleavable by an enzyme that specifically cleaves a uracil base from a polynucleotide, such as uracil DNA glycosylase (UDG) enzyme. As another example, the at least one cleavable base 130 may include at least one uracil base, and may be cleaved by a UDG enzyme in conjunction with an Endonuclease VIII (EndoVIII) or Endonuclease IV (EndoIV) enzyme. Cleaving the at least one cleavable base 130 may include contacting the at least one cleavable base 130 to a solution including UDG enzyme. The solution including UDG enzyme may be contacted to the at least one cleavable base 130 by being brought into contact with the complex 150 including the surface-bound oligonucleotide 120, the at least one cleavable base 130, and the product oligonucleotide 140, and/or into contact with the surface 110.

The solution may include about 100 μL, about 200 μL, about 300 μL, about 400 μL, about 500 μL, about 1 mL, about 10 mL, about 20 mL, about 50 mL, about 100 mL, about 500 mL, about 1 L, about 10 L, about 20 L, about 50 L, about 100 L, or more of a UDG mixture containing UDG enzyme and a buffer. The buffer may contain Tris-HCl, dithiothreitol (DTT), and ethylenediaminetetraacetic acid (EDTA), and may include a basic pH, such as a pH of about 7.1, about 7.2, about 7.3, about 7.4, about 7.5, about 7.6, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, or more, inclusive. Contacting the complex 150 to a solution including UDG enzyme may further include contacting the complex 150 to the solution for a duration of about 1 minute to about 60 minutes, such as about 1 minute, about 2 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. For example, the duration may be about 30 minutes. Contacting the complex 150 to a solution including UDG enzyme may further include contacting the complex 150 to the solution at a heated temperature, such as a temperature of about 20 degrees Celsius to about 45 degrees Celsius, such as about 20 degrees Celsius, about 25 degrees Celsius, about 30 degrees Celsius, about 40 degrees Celsius, or about 45 degrees Celsius, inclusive. For example, the solution may have a temperature of about 37 degrees Celsius. Cleaving of the at least one cleavable base 130 by the UDG enzyme may leave an abasic site in the complex 150 where the at least one cleavable base 130 was located, between the surface-bound oligonucleotide 120 and the product oligonucleotide 140.

After contacting the complex 150 to the solution comprising UDG enzyme, the complex 150 and the surface 110 may be washed. Washing may include contacting the surface 110 with a washing reagent, such as water. For example, the surface 110 may be contacted to about 10 μL to about 1 L or more of washing reagent, such as about 10 μL, about 20 μL, about 50 μL, about 100 μL, about 200 μL, about 300 μL, about 400 μL, about 500 μL, about 600 μL, about 700 μL, about 800 μL, about 900 μL, about 1 mL, about 2 mL, about 5 mL, about 10 mL, about 20 mL, about 50 mL, about 100 mL, about 200 mL, about 500 mL, about 1 L, or more. After contacting the surface 110 to a washing reagent, the surface 110 may be allowed to dry or may be vacuum dried.

After drying the surface 110, the complex 150 may be contacted to, contacted by, or exposed to an enzyme or compound capable of breaking phosphodiester bonds at the abasic site where the at least one cleavable base was located. For example, the complex 150 may be contacted to about 10 μL to about 1 L or more of an AMA mixture containing a 1:1 (v/v) solution of 30% ammonium hydroxide and 40% aqueous methylamine, such as about 10 μL, about 20 μL, about 50 μL, about 100 μL, about 200 μL, about 300 μL, about 400 μL, about 500 μL, about 600 μL, about 700 μL, about 800 μL, about 900 μL, about 1 mL, about 2 mL, about 5 mL, about 10 mL, about 20 mL, about 50 mL, about 100 mL, about 200 mL, about 500 mL, about 1 L, or more, inclusive. Contacting the complex 150 to the AMA mixture may further include contacting the complex 150 to the AMA mixture for a duration of about 1 minute to about 60 minutes, such as about 1 minute, about 2 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. For example, the duration may be about 30 minutes.

Contacting the complex 150 to the AMA mixture may further include contacting the complex 150 to the AMA mixture at a heated temperature, such as a temperature of about 20 degrees Celsius to about 45 degrees Celsius, such as about 20 degrees Celsius, about 25 degrees Celsius, about 30 degrees Celsius, about 40 degrees Celsius, or about 45 degrees Celsius, inclusive. For example, the AMA mixture may have a temperature of about 37 degrees Celsius. Contacting the complex 150 to the AMA mixture may release the product oligonucleotide 140 from the surface-bound oligonucleotide 120. The complex 150 may be contacted with an AMA mixture two or more times. For example, after contacting the complex 150 to the AMA mixture, the AMA mixture may be removed from the surface 110 and collected in a container, and the complex 150 may be contacted to a second AMA mixture as described herein after which the second AMA mixture may be removed from the surface 110 and collected in the same container as or a different container from the first collected AMA mixture.

As another example, the at least one cleavable base 130 may include at least one inosine base, and may be cleavable by an enzyme that cleaves at the site of an inosine base, such as Endonuclease V (EndoV) enzyme. In some embodiments, the at least one cleavable base 130 includes a linker that includes an uracil base and/or an inosine base. Cleaving the at least one cleavable base 130 may include contacting the at least one cleavable base 130 to a solution including EndoV enzyme. The solution may include about 100 μL, about 200 μL, about 300 μL, about 400 μL, about 500 μL, about 1 mL, about 10 mL, about 20 mL, about 50 mL, about 100 mL, about 500 mL, about 1 L, about 10 L, about 20 L, about 50 L, about 100 L, inclusive, or more of a solution including EndoV enzyme and a buffer. The buffer may contain 50 mM potassium acetate, 20 mM Tris-acetate, 10 mM magnesium acetate, and 1 mM DTT, and may include a basic pH, such as a pH of about 7.1, about 7.2, about 7.3, about 7.4, about 7.5, about 7.6, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, or more.

Contacting the complex 150 to a solution including EndoV enzyme may further include contacting the complex 150 to the solution for a duration of about 1 minute to about 60 minutes, such as about 1 minute, about 2 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes, inclusive. For example, the duration may be about 30 minutes. Contacting the complex 150 to a solution including EndoV enzyme may further include contacting the complex 150 to the solution at a heated temperature, such as a temperature of about 20 degrees Celsius to about 45 degrees Celsius, such as about 20 degrees Celsius, about 25 degrees Celsius, about 30 degrees Celsius, about 40 degrees Celsius, or about 45 degrees Celsius, inclusive. For example, the solution may have a temperature of about 37 degrees Celsius. Cleaving of the at least one cleavable base 130 by the EndoV enzyme may release the product oligonucleotide 140 from the surface-bound oligonucleotide 120.

As used herein, the terms “Endonuclease V”, “EndoV”, and “EndoV enzyme” may refer to an enzyme that binds to a polynucleotide and cleaves the polynucleotide immediately downstream (i.e., a 3′ end) of a deoxyriboinosine base or a riboinosine base. Cleaving of the at least one cleavable base 130 by the EndoV enzyme may result in the at least one cleavable base 130 being removed from the surface-bound oligonucleotide 120, or being removed from the product oligonucleotide 140, or being removed from the surface-bound oligonucleotide 120 and from the product oligonucleotide 140. For example, the at least one cleavable base 130 may be cleaved at its bond to surface-bound oligonucleotide 120, or the at least one cleavable base 130 may be cleaved at its bond to the product oligonucleotide 140, or the at least one cleavable base 130 may be cleaved at its bond to the surface-bound oligonucleotide 120 and cleaved at its bonds to the product oligonucleotide 140. Alternatively, cleavage may result in a portion of the at least one cleavable base 130 being removed from the surface-bound oligonucleotide 120. For example, the at least one cleavable base 130 may include two or more cleavable bases, and cleavage may result in one or more cleavable bases being removed from the surface-bound oligonucleotide 120 and one or more cleavable bases being retained on the surface-bound oligonucleotide 120. As another example, the at least one cleavable base 130 may include two or more cleavable bases, and cleavage may result in one or more cleavable bases being removed from the product oligonucleotide 140 and one or more cleavable bases being retained on the product oligonucleotide 140.

The released product oligonucleotide 140 may be extracted. Extraction may be performed after cleavage by retrieving solution in contact with the surface 110 and the product oligonucleotide 140. For example, after contacting the complex 150 with a solution including UDG enzyme and then an AMA mixture. The AMA mixture may contain at least some of released product oligonucleotide 140. The AMA mixture containing at least some of the released product oligonucleotide 140 may be removed from the surface 110 and collected in a container. The complex 150 may be contacted with one or more additional AMA mixtures in a similar manner as contact with the first AMA mixture. After contact to complex 150, each of the one or more additional AMA mixtures may be removed from the surface 110 and collected in a container. The container may be the same container containing the first collected AMA mixture, or it may be a different container.

Extraction may be aided by assembling a chip including the surface 110 in an extraction device. The extraction device may include a metal bottom, and may be covered or uncovered. The extraction device may be heated, such as by placement in a thermocycler, to a temperature of about 20 degrees Celsius to about 45 degrees Celsius, such as about 20 degrees Celsius, about 25 degrees Celsius, about 30 degrees Celsius, about 35 degrees Celsius, about 40 degrees Celsius, or about 45 degrees Celsius, inclusive. For example, the extraction device may be heated to a temperature of about 37 degrees Celsius. Retrieved solution containing product oligonucleotide 140 after each incubation step may be added together, or may be kept separate. Retrieved solution containing product oligonucleotide 140 may be dried, such as vacuum-dried.

After cleavage, the surface-bound oligonucleotide 120 may be regenerated for enzymatic DNA synthesis. In some embodiments, regenerating the surface-bound oligonucleotide 120 may include removing a 3′-PO₄ phosphate group at the 3′ end of the surface-bound oligonucleotide 120. The 3′-PO₄ phosphate group may be removed enzymatically. For example, T4 polynucleotide kinase (PNK) enzyme may be used to remove the 3′-PO₄ phosphate group from the 3′ end of the surface-bound oligonucleotide 120. After removal of the phosphate group, the 3′ end of the surface-bound oligonucleotide 120 contains a 3′-OH hydroxyl group and may be available for use in another round of oligonucleotide synthesis. In some embodiments, a second oligonucleotide containing at least one cleavable base may be added to the 3′ end of the surface-bound oligonucleotide. After addition to the surface-bound oligonucleotide, the second oligonucleotide containing at least one cleavable base may be available for use in another round of oligonucleotide synthesis. For example, a uracil-containing oligonucleotide may be added to the surface-bound oligonucleotide at its 3′ end.

Further provided are various devices and systems for carrying out the methods described herein. For example, provided herein are chips with functionalized surfaces for carrying out one or more rounds of enzymatic DNA synthesis.

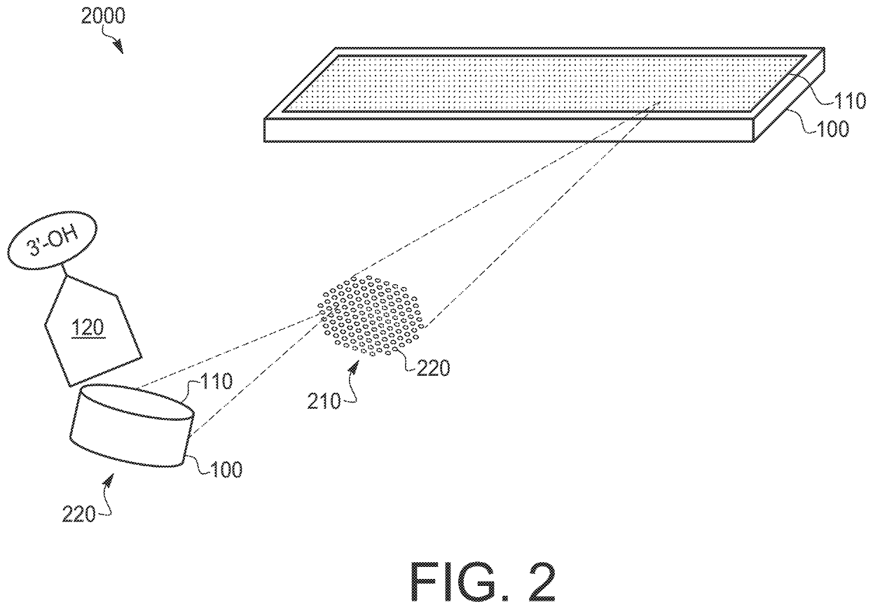

Referring now to FIG. 2, described herein is an exemplary chip 2000 configured to perform the methods and processes described above. The chip 2000 may include a base 100 and a surface 110. The surface 110 may be functionalized. The surface 110 may include at least one cluster 210 of wells 220. Each well 220 may include a surface-bound oligonucleotide 120, having a 3′-OH hydroxyl group at its 3′ end.

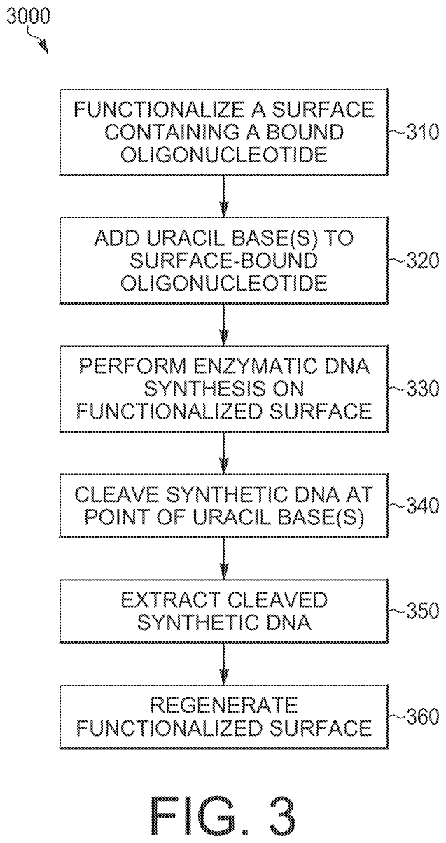

Referring now to FIG. 3, described herein is an exemplary embodiment 3000 of the disclosed methods, for enzymatic synthesis and cleavage of a synthesized oligonucleotide at the site of at least one uracil base. At 310, a substrate including a surface containing a bound oligonucleotide is functionalized. The surface may include silicon, silicon dioxide, silicon nitride, a metal (e.g., platinum, gold, indium tin oxide), a conductive material, a non-conductive material, beads, plastic, a polymer with functional groups for attachment, a polystyrene support, or a composite surface. The surface may be functionalized using any known functionalization chemistry to enable or facilitate binding of an oligonucleotide to the surface. For example, the surface may be a silicon surface functionalized with silane. The surface may be planar or it may include geometrical features as desired (e.g., nano-wells, electrodes, porous regions, etc.).

At 320, at least one uracil base may be added to the surface-bound oligonucleotide. The at least one uracil base may be added to the surface-bound oligonucleotide by an enzyme. For example, the surface-bound oligonucleotide may be contacted with a mixture containing one or more enzymes, such as TdT, cytosine deaminase, cytosine deamination, dUTP, variants of TdT, or any combination thereof. TdT or TdT variants may be provided with or without solubility tags. In some embodiments, one uracil base may be added to the surface-bound oligonucleotide. In some embodiments, more than one (e.g., 2, 3, 4, 5, 6, 7, 8, 9, 10, etc.) uracil bases may be added to the surface-bound oligonucleotide. In some embodiments, the at least one uracil base may be protected or unprotected.

At 330, enzymatic DNA synthesis may be performed on the functionalized surface to produce synthetic DNA molecules. Enzymatic DNA synthesis may be performed according to various techniques known within the art.

At 340, the synthetic DNA molecule(s) may be cleaved at the point of the uracil base(s). The synthetic DNA molecule added to a surface-bound oligonucleotide containing at least one uracil base may be cleaved from the surface-bound oligonucleotide at the site of the at least one uracil base. For example, the synthetic DNA molecule may be cleaved by an enzyme, such as UDG enzyme, at the site of the at least one uracil base. In some embodiments, the synthetic DNA molecule may be contacted (incubated) with UDG enzyme, such as UDG enzyme in buffer. For example, the synthetic DNA molecule may be incubated with UDG enzyme for about 1 minute, about 2 minutes, about 5 minutes, about 10 minutes, about 15 minutes, about 20 minutes, about 25 minutes, about 30 minutes, about 40 minutes, about 50 minutes, about 1 hour, about 2 hours, inclusive, or more. The synthetic DNA molecule may be incubated with UDG enzyme at about 20 degrees Celsius to about 40 degrees Celsius, about 30 degrees Celsius to about 40 degrees Celsius, about 35 degrees Celsius to about 40 degrees Celsius, or about 37 degrees Celsius. In some embodiments, a thermocycler may be used for incubation of the synthetic DNA molecule. For example, a thermocycler may be used to incubate the synthetic DNA molecule with UDG enzyme for about 30 minutes at about 37 degrees Celsius.

In some embodiments, cleaving the synthetic DNA molecule may include incubation with UDG enzyme and/or incubation with an AMA mixture containing a 1:1 (v/v) solution of 30% ammonium hydroxide and 40% aqueous methylamine. For example, cleaving the synthetic DNA molecule may include incubation with UDG enzyme followed by at least one incubation with an AMA mixture. In some embodiments, the synthetic DNA molecule may be incubated with an AMA mixture for about 1 minute, about 2 minutes, about 5 minutes, about 10 minutes, about 15 minutes, about 20 minutes, about 25 minutes, about 30 minutes, about 40 minutes, about 50 minutes, about 1 hour, about 2 hours, inclusive, or more. The synthetic DNA molecule may be incubated with the AMA mixture at about 20 degrees Celsius to about 40 degrees Celsius, about 30 degrees Celsius to about 40 degrees Celsius, about 35 degrees Celsius to about 40 degrees Celsius, or about 37 degrees Celsius. In some embodiments, a thermocycler may be used for incubation of the synthetic DNA molecule. For example, a thermocycler may be used to incubate the synthetic molecule with AMA mixture for about 30 minutes at about 37 degrees Celsius. In some embodiments, the at least one incubation with AMA mixture may include one incubation with AMA mixture. In some embodiments, the at least one incubation with AMA mixture may include more than one (e.g., 2, 3, 4, 5, 6, 7, 8, 9, 10, or more) incubations with AMA mixture.

At 350, the synthetic DNA molecule(s) may be extracted. Extraction may be performed by retrieving solution in contact with the surface after cleaving. For example, solution may be retrieved after incubation with UDG enzyme. Solution may be retrieved again after at least one incubation with AMA mixture. Extract may be aided by assembling a chip including the surface in an extraction device. The extraction device may contain a metal bottom, and may be covered or uncovered. The extraction device may be heated, such as by placement in a thermocycler. Retrieved solution after each incubation step may be added together, or may be kept separate. Samples from the retrieved solution may include extracted, cleaved synthetic DNA molecule(s), and may be dried, such as vacuum-dried.

At 360 the functionalized surface may be regenerated for enzymatic DNA synthesis. For example, a 3′-PO₄ phosphate group may be enzymatically removed from the 3′ end of the surface-bound oligonucleotide. For example, the 3′-PO₄ phosphate group may be removed by contact (incubation) with T4 PNK enzyme. After removal of the phosphate group, the 3′ end of the surface-bound oligonucleotide contains a 3′-OH hydroxyl group and may be available for use in another round of enzymatic DNA synthesis.

As another example of regenerating the functionalized surface, a second uracil-containing oligonucleotide may be ligated to the surface-bound oligonucleotide. For example, a second uracil-containing oligonucleotide may be added to the 3′ end of the surface-bound oligonucleotide using TdT, variants of TdT, cytosine deaminase, cytosine deamination, dUTP, or any combination thereof. Such enzymes may be provided with or without solubility tags. After addition of the second uracil-containing oligonucleotide, the surface-bound oligonucleotide may be available for use in another round of enzymatic DNA synthesis.

As another example of regenerating the functionalized surface, the 3′-PO₄ phosphate group may be removed and a second uracil-containing oligonucleotide may be ligated to the surface-bound oligonucleotide. For example, the 3′-PO₄ phosphate group may be removed by contact (incubation) with T4 PNK enzyme. After removal of the phosphate group, the 3′ end of the surface-bound oligonucleotide contains a 3′-OH hydroxyl group and may be available for ligating a second uracil-containing oligonucleotide using TdT, variants of TdT, cytosine deaminase, cytosine deamination, dUTP, or any combination thereof. Such enzymes may be provided with or without solubility tags.

### EXAMPLES

### Example 1: In-Solution Cleavage of Oligonucleotides at Cleavable Uracil Base

A 45-mer oligonucleotide having 45 bases was provided in solution, the 45-mer oligonucleotide having the nucleic acid sequence of:

(SEQ ID NO: 1)
5′ TTTTTTTTTTTTTTUTGCAATGCAGTACCGAGATCAGTCAGTGTC
3′.

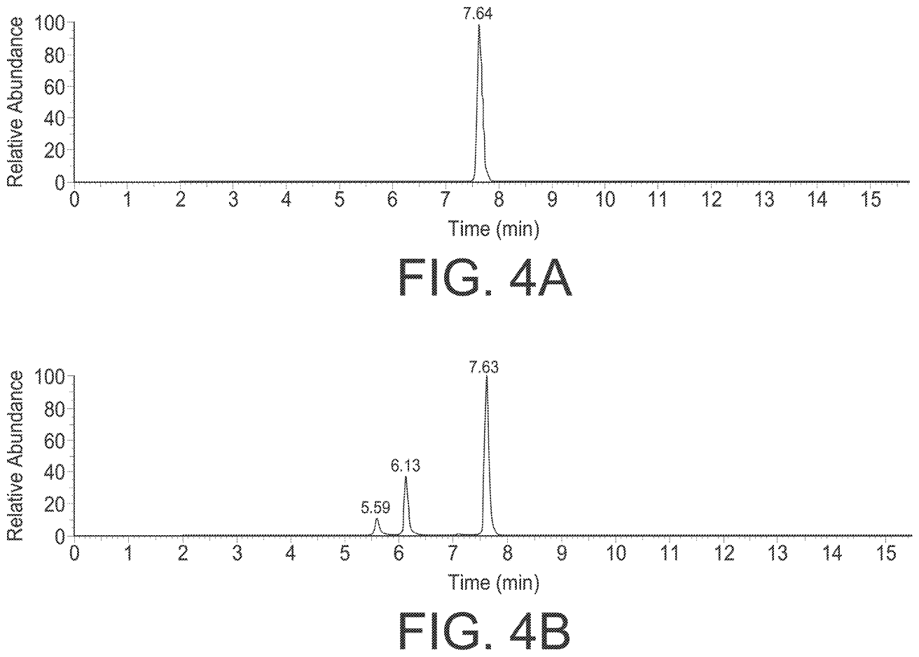

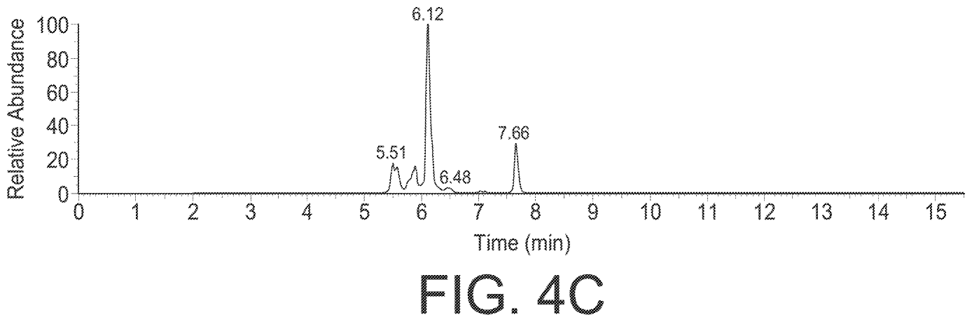

A sample of the solution including the 45-mer oligonucleotide was analyzed using liquid chromatography-mass spectrometry (LC-MS). The chromatogram generated from the LC-MS analysis of the solution including the 45-mer oligonucleotide is shown in FIG. 4A.

As shown in FIG. 4A, LC-MS results demonstrated a single peak of relatively high abundance at around 7.6 minutes in the sample containing only the 45-mer oligonucleotide. Thus, the peak at around 7.6 minutes corresponded to the intact 45-mer oligonucleotide.

Next, a UDG enzyme was added to a solution containing the 45-mer oligonucleotide to excise the uracil base at position 15 from the 5′ end in the 45-mer sequence (SEQ ID NO: 1). Fifty μL of solution containing 20 μL of UDG enzyme, 28.2 μL of 1×UDG reaction buffer (20 mM Tris-HCl, 1 mM DTT, 1 mM EDTA, pH 8), and 1.8 μL of 100 μM stock concentration of the 45-mer oligonucleotide was prepared and allowed to incubate for 30 minutes at 37 degrees Celsius. After incubation, the sample was purified to remove the enzyme from the mixture, vacuum-dried, and analyzed using LC-MS. The chromatogram generated from the LC-MS of the sample is shown in FIG. 4B, demonstrating three peaks of relatively high abundance at around 7.6 minutes (highest peak), 6.1 minutes, and 5.5 minutes. The peak around 7.6 minutes (representing the 45-mer oligonucleotide with an abasic site at position 15) was the highest peak, indicating the 45-mer oligonucleotide was relatively more abundant than the shorter molecules represented by the two new peaks. The new peak at 5.5 minutes represented the 14-mer fragment of fourteen thymine bases, corresponding to base positions 1-14 from the 5′ end of the 45-mer oligonucleotide. The new peak at around 6.1 minutes represented the 30-mer fragment of 30 bases corresponding to base positions 16-45 of the 45-mer oligonucleotide. This fragmentation of the 45-mer oligonucleotide was likely due to cleavage at the abasic site during the post-processing steps and the analysis process.

Next, an ammonia-methylamine (AMA) mixture including a 1:1 (v/v) solution of 30% ammonium hydroxide and 40% aqueous methylamine was added to a solution containing the 45-mer oligonucleotide which had been incubated with UDG enzyme, in order to cleave the 45-mer oligonucleotide at the abasic site at position 15 generated by the UDG enzyme. The new mixture of UDG reaction along with AMA was incubated for one hour at room temperature in the sealed tube. A This sample of the collected AMA mixtures was vacuum-dried to remove volatile components of the AMA and analyzed via LC-MS, and the chromatogram generated from the LC-MS is shown in FIG. 4C. As shown in FIG. 4C, LC-MS results demonstrated at least three peaks of relatively high abundance at around 5.5 minutes (the 14-mer fragment), about 6.1 minutes (the 30-mer fragment), and about 7.6 minutes (the 45-mer oligonucleotide). After addition of the AMA mixture, the highest peak was that of the peak at around 6.1 minutes, representing the 30-mer fragment. Thus, the addition of AMA mixture lead to the 30-mer fragment being relatively more abundant than the 45-mer oligonucleotide, indicating increased cleavage of the 45-mer oligonucleotide at position 15.

### Example 2: In-Solution Cleavage of Oligonucleotides at Cleavable Uracil Base

The methods described above were repeated for a second oligonucleotide, a 48-mer oligonucleotide having 48 bases and the nucleic acid sequence of:

(SEQ ID NO: 2)
5′ TTTTTTTTTTTTTTUUUUTGCAATGCAGTACCGAGATCAGTCAGTG
TC 3′.

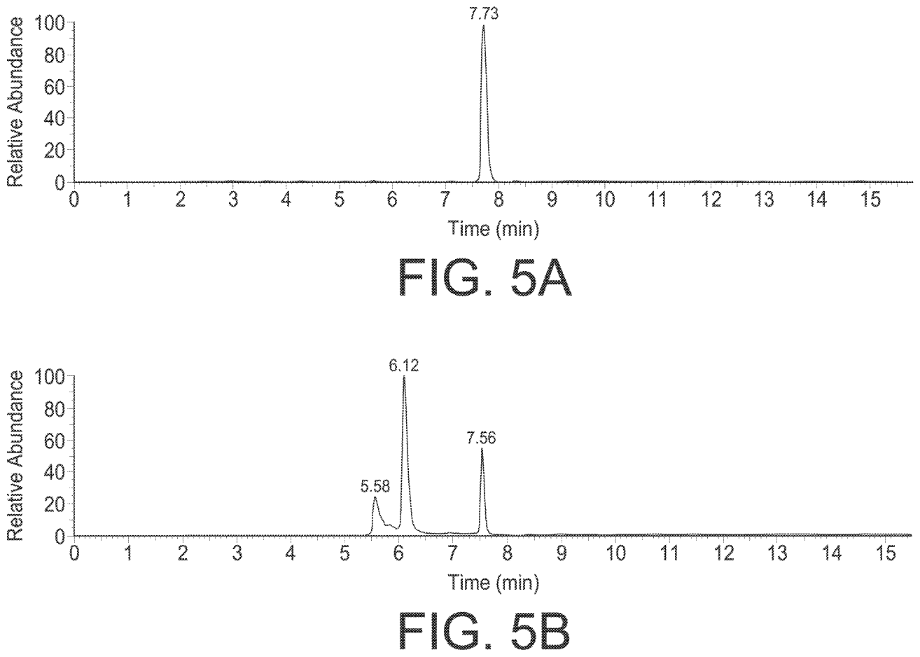

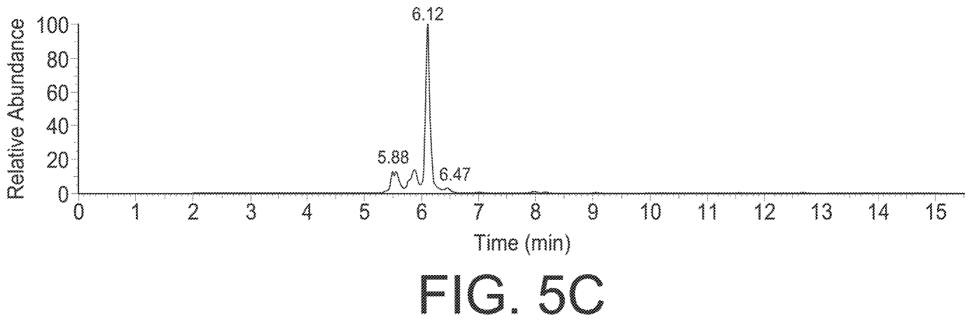

A solution including the 48-mer oligonucleotide was analyzed using LC-MS, and the resulting chromatogram generated is shown in FIG. 5A. As shown in FIG. 5A, LC-MS results demonstrated a single peak of relatively high abundance at around 7.7 minutes in the sample containing only the 48-mer oligonucleotide. Thus, the peak at around 7.7 minutes corresponded to the intact 48-mer oligonucleotide. Next, the UDG enzyme mixture described in Example 1 was added to the solution including the 48-mer oligonucleotide, in order to excise the uracil bases at positions 15-18 in the 48-mer sequence (SEQ ID NO: 2). The resulting mixture was incubated for 30 minutes at 37 degrees Celsius. After incubation, a sample from the solution was collected, vacuum-dried, and analyzed using LC-MS. The chromatogram generated from the LC-MS of the sample is shown in FIG. 5B, demonstrating three peaks of relatively high abundance at around 7.5 minutes, around 6.1 minutes (highest peak), and around 5.5 minutes. The peak around 7.5 minutes corresponded to the 48-mer oligonucleotide with abasic sites at positions 15-18. The peak at around 6.1 minutes corresponding to a 30-mer fragment of 30 bases corresponding to base positions 19-48 of the 48-mer sequence. The peak at around 5.5 minutes corresponded to a 14-mer fragment of fourteen thymine bases at positions 1-14 of the 48-mer sequence. This fragmentation of the 48-mer oligonucleotide was likely due to cleavage at the abasic site during the post-processing and the analysis process.

Next, an AMA mixture as described in Example 1 was added to a solution containing the 48-mer oligonucleotide which had been incubated with UDG enzyme, in order to cleave the 48-mer oligonucleotide at the abasic sites at positions 15-18 generated by the UDG enzyme. First, the solution was incubated with UDG enzyme as described above. Then, a 1:1 v/v AMA mixture was added to the sample containing UDG reaction, which was then incubated for one hour at room temperature. This sample was vacuum-dried and analyzed via LC-MS, and the chromatogram generated from the LC-MS is shown in FIG. 5C. As shown in FIG. 5C, the LC-MS results demonstrated at least two peaks of relatively high abundance at around 5.5 minutes (the 14-mer fragment) and 6.1 minutes (the 30-mer fragment). In contrast to the sample from the solution that was solely incubated with UDG enzyme, the sample incubated with UDG enzyme and AMA mixture showed no peak at around 7.5 minutes, indicating that all or most of the 48-mer oligonucleotide was cleaved.

Comparing the results of Example 1 with the present Example 2, it is noted that the present Example 2, which analyzed an oligonucleotide containing multiple uracil bases, demonstrated increased cleavage as compared to Example 1, which analyzed an oligonucleotide containing one uracil base.

The results therefore demonstrate the successful cleavage of oligonucleotides into fragments at the site of multiple uracil bases by UDG enzyme and AMA mixture. The results further demonstrate that increasing the number of uracil bases from one to four also increased cleavage activity.

### Example 3: Incorporation of Cleavable Layer on Functionalized Surface of Chip

An exemplary chip was prepared with a uracil-containing oligonucleotide on a surface. A variant of TdT enzyme (“NTT3”) was used to incorporate an oligomer sequence including an unprotected uracil base (U) and ten consecutive thymine bases, i.e., 5′ UTTTTTTTTTT 3′ (SEQ ID NO: 3) by extending a template oligonucleotide having the sequence 5′ TTTTTTT-PCL-TTTTTTTTTTT 3′ (SEQ ID NO: 4), PCL being a photo-cleavable linker which, upon exposure to ultraviolet light having a wavelength of 365 nm, cleaved to release the fragment having the sequence 5′ Phos-TTTTTTTTTTT 3′ (SEQ ID NO: 5). The released fragment was collected, while the fragment having the sequence 5′ TTTTTTT-Phos 3′ was retained on the surface. Due to the inclusion of the unprotected uracil base, multiple consecutive uracil bases were established on the surface, and ten cycles of base addition of protected thymine bases were performed using the following methods to generate the controlled oligonucleotide having ten thymine bases on the cleavable layer.

A deblocking step was performed to remove the protecting group on the 3′-OH terminus of the surface-bound oligonucleotide using a reagent. A deblocking reagent may be referred to as “chem-deblock” when chemically removing all groups, or referred to as “e-deblock” when removing the deblocking group by electrochemically producing the acid in specific regions of the surface addressed via electrodes. Here, a chem-deblock reagent was used of which a 1,000 mL stock solution included about 82.03 g sodium acetate and about 48.3 g sodium nitrate in water. A coupling/extension step was performed to add a protected nucleotide base (alternatively, an unprotected nucleotide base could be added in some cases) to the deblocked 3′-OH terminus of the surface-bound oligonucleotide using TdT enzyme, and by flowing and incubating a coupling mixture over the surface. The coupling mixture was used of which a 5,200 mL stock solution included 232.2 mL of 111.9 M TdT enzyme, 260 mL of 10 mM nucleotide, 520 mL of 10×NTT reaction buffer, 520 mL of 2.5 mM MnCl₂, 5.2 mL of 10% w/v Tween-20, and 3666.2 mL water. A surface renewal step was performed by flowing 15 mL of a solution including Proteinase K over the surface to remove, via digestion, TdT enzyme bound to the surface. The Proteinase K solution included 0.075 mL of 0.05 mg/mL Proteinase K (Sigma). Each of the above steps was followed by a washing step using a wash reagent to remove reagents from the surface. The wash reagent included 50 mM HEPES buffer, 0.1% w/v Tween-20, and 1 M sodium hydroxide in water. The steps were then repeated for ten cycles, after which the surface was washed with water and dried before it was removed from the flowcell.

After generation of the controlled oligonucleotide on the cleavable layer, the template containing the synthesized oligonucleotide was extracted from the surface by exposing the photo-cleavable linker in the template to ultraviolet light. This generated the extracted material of 5′ Phos-TTTTTTTTTTT(U)xTTTTTTTTTT 3′ (SEQ ID NO: 6), where (U)ₓ represents the variable number of uracil bases incorporated by the TdT enzyme. Mass spectrometry was performed to evaluate the presence of the uracil-containing oligonucleotide on the surface.

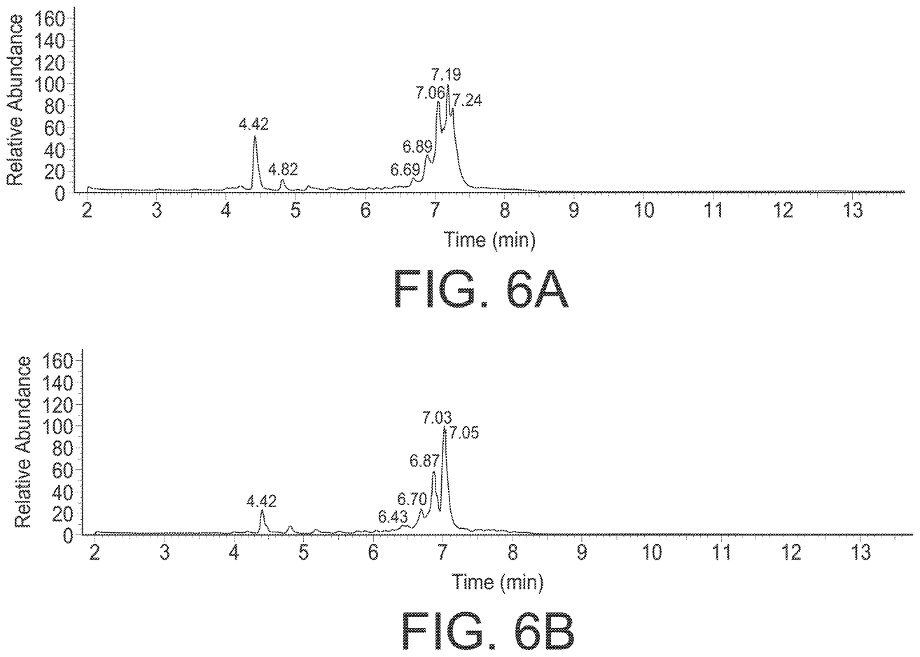

As shown in FIG. 6A, after 11 cycles of incorporation, mass spectrometry demonstrated a peak of relatively high abundance at around 4.4 minutes and a higher peak at around 6.6-7.2 minutes. As a control, as shown in FIG. 6B, a sequence of just ten thymine bases, i.e. TTTTTTTTTT (SEQ ID NO: 7) was also incorporated, with mass spectrometry demonstrating a similar pattern of peaks at 4.4 minutes and 6.8-7.0 minutes. Thus, the 11-mer sequence was successfully incorporated into the cleavable layer on the chip. The fragment of the template strand which was not incorporated corresponded to the peak detected at 4.4 minutes, and the fragments which demonstrated successful incorporation of multiple uracil bases and ten thymine bases corresponded to the peaks detected around 6.8-7.0 minutes.

### Example 4: On-Chip Cleavage of Synthetic Oligonucleotide at Cleavable Uracil Base

An exemplary chip was prepared with an uracil-containing oligonucleotide on a surface, according to the methods described above in Example 3.

Enzymatic DNA synthesis was performed to synthesize a desired oligonucleotide added to the surface-bound oligonucleotide containing a cleavable linker as shown: 5′ TTTTTTT-PCL-TTTTTTTTTT(U)xTTTTTTTTTT 3′ (SEQ ID NO: 8),where “PCL” indicates a photocleavable linker and (U)ₓ represents the variable number of incorporated uracil bases.

Next, the chip was removed from a flowcell chamber and subjected to a UDG reaction in a separate apparatus.

After performance of enzymatic synthesis, the chip was assembled in an extraction device containing a metal bottom. The extraction device was pre-heated to a temperature of 37 degrees Celsius by placement in a thermocycler for approximately 30 minutes at 37 degrees Celsius, with a lid temperature of 42 degrees Celsius.

Next, 200 μL of a uracil DNA glycosylase (UDG) mixture containing 20 μL of UDG enzyme and 180 μL 1× buffer containing 20 mM Tris-HCl, 1 mM DTT, 1 mMEDTA, pH 8 (UDG reaction buffer) was added to the prepared chip. The chip containing the UDG mixture was swirled to ensure coverage of all active sites on the chip, and the top of the extraction device was covered to avoid evaporation of the reaction liquids. Next, the assembly was placed in a thermocycler and incubated with the UDG mixture for 30 minutes at 37 degrees Celsius. After incubation, the assembly was removed from the thermocycler and uncovered. The UDG mixture was retrieved from within the assembly and set aside for testing.

Next, 400 μL of water was added to the used chip, and the chip containing the water was swirled to ensure washing of all active sites. Water from within the assembly was retrieved and vacuum dried. Ultraviolet absorption amounts of the nanodrop samples of the retrieved water and retrieved UDG mixture were measured. The chip was allowed to dry before proceeding to the next step.

Next, 400 μL of an AMA mixture containing a 1:1 (v/v) solution of 30% ammonium hydroxide and 40% aqueous methylamine was added to the dried chip. The assembly was allowed to incubate with the AMA mixture for approximately 10, 15, and 20 minutes at room temperature for separate cases. After incubation, the AMA mixture was retrieved and set aside. An additional 400 μL of AMA was added to the chip, and allowed to incubate for approximately 10, 15, and 20 minutes at room temperature for separate cases. After the second AMA incubation, the AMA mixture was retrieved and added to the previously-retrieved AMA mixture. A sample of the retrieved AMA mixture, containing cleaved oligonucleotides, was collected and vacuum-dried. An ultraviolet-visible (UV-vis) detector was used to detect intensity and absorbance of ultraviolet and visible light of compounds in the dried AMA mixture sample.

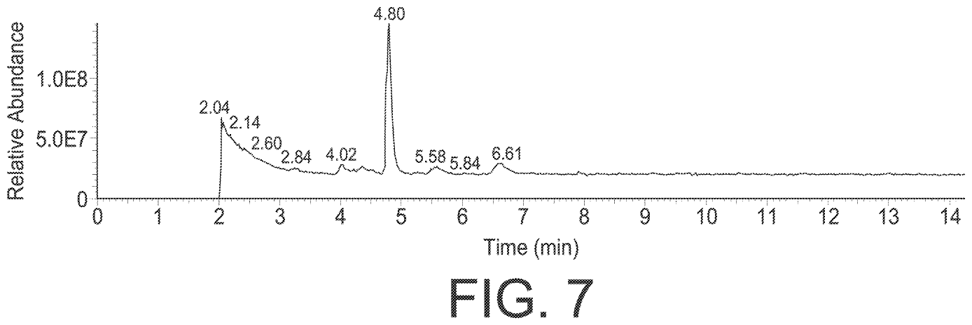

As shown in FIG. 7, UV-vis results demonstrated that intensity peaked at around 4.8 minutes, and the mass confirmed the presence of the oligonucleotide containing ten thymine bases which were incorporated upstream of the cleavable layer of uracil base.

### Example 5: Two Rounds of On-Chip Cleavage of Oligonucleotides at Cleavable Base Site

The following example will demonstrate two rounds of successful synthesis and enzymatic cleavage of oligonucleotides on the same exemplary chip.

The methods described above in Example 4 are repeated twice to prepare an exemplary chip and produce two oligonucleotides consecutively synthesized on the same chip. Samples are collected from each of the two oligonucleotides and tested with a UV-vis detector to identify compounds contained therein. Absorbance peaks indicate successful synthesis and enzymatic cleavage of both oligonucleotides. The second oligonucleotide is synthesized and enzymatically cleaved at rates comparable to the first oligonucleotide.

In some examples, the methods described above in Example 4 can be repeated twice in combination with a phosphatase repair process between the two rounds of synthesis. Where inosine is used as the cleavable base and Endo V is used as the cleaving enzyme, the two rounds of synthesis can be performed consecutively without a phosphatase repair step between the two rounds of synthesis.

### Example 6: Many Rounds of On-Chip Cleavage of Oligonucleotides at Cleavable Base Site

The following example will demonstrate multiple rounds of successful synthesis and enzymatic cleavage of oligonucleotides on the same exemplary chip.

The methods described above in Example 4 are repeated multiple times to prepare an exemplary chip and produce multiple oligonucleotides consecutively synthesized on the same chip. Samples are collected from each of the oligonucleotides and tested with a UV-vis detector to identify compounds contained therein. Absorbance peaks indicate successful synthesis and enzymatic cleavage of most or all of the oligonucleotides. Most or all of the oligonucleotides are synthesized and enzymatically cleaved at comparable rates.

### Example 7: On-chip Cleavage of Oligonucleotides at Inosine Base Site

The following example will demonstrate successful enzymatic cleavage of synthetic oligonucleotides from surface-bound oligonucleotides containing a cleavable linker including one or more inosine bases.

An exemplary chip is prepared with an oligonucleotide containing a cleavable linker including an inosine base on a surface. An enzyme is used to incorporate an inosine base into a cleavable layer on the chip. Enzymatic DNA synthesis can be performed to synthesize a desired oligonucleotide. Mass spectrometry confirms that the inosine base is successfully incorporated into the cleavable layer. Mass spectrometry can also confirm the sequence of the synthesized desired oligonucleotide.

The chip is assembled in an extraction device containing a metal bottom. The extraction device is heated to a temperature of 37 degrees Celsius by placement in a thermocycler for approximately 30 minutes at 37 degrees Celsius, with a lid temperature of 42 degrees Celsius.

Endonuclease V (EndoV) enzyme mixture containing EndoV enzyme and 1× buffer containing 50 mM potassium acetate, 20 mM Tris-acetate, 10 mM magnesium acetate, 1 mM DTT, pH 7.9 (NEBuffer™ 4) is added to the prepared chip. The chip containing the EndoV enzyme mixture is swirled to ensure coverage of all active sites on the chip, and the top of the extraction device is covered. Next, the assembly is placed in a thermocycler and incubated with the EndoV enzyme mixture. After incubation, the assembly is removed from the thermocycler and uncovered. The EndoV enzyme mixture is retrieved from within the assembly and set aside for testing.

Next, water is added to the used chip, and the chip containing the water is swirled to ensure washing of all active sites. Water from within the assembly is retrieved and added to the tube with the EndoV enzyme mixture and vacuum-dried, and nanodrop samples of the retrieved water and retrieved EndoV enzyme mixture are collected and vacuum-dried. A purification reaction may be performed to remove any remaining enzyme. An ultraviolet-visible (UV-vis) detector is used to detect intensity and absorbance of ultraviolet and visible light of compounds in the dried samples.

UV-vis results demonstrate peaks of intensity and absorbance representing an intact oligonucleotide and representing fragments corresponding to portions of the oligonucleotide separated by the cleavable linker. The peaks representing the fragments are relatively higher than the peak representing the intact oligonucleotide, indicating successful cleavage of most of the oligonucleotide.

### Example 8: In-solution Demonstration of Reuse

The present Example describes the steps of cleaving, repairing, and extending the same site on an oligonucleotide, with the expected reaction products observed at each step via LC-MS, and demonstrates reuse of an oligonucleotide in solution.

A 32-mer oligonucleotide having 32 bases was provided in solution, the 32-mer oligonucleotide having the nucleic acid sequence of:

(SEQ ID NO: 9)
5′ TTTTTTTTTTTTTTTTTTTTUTTTTTTTTTTT 3′.

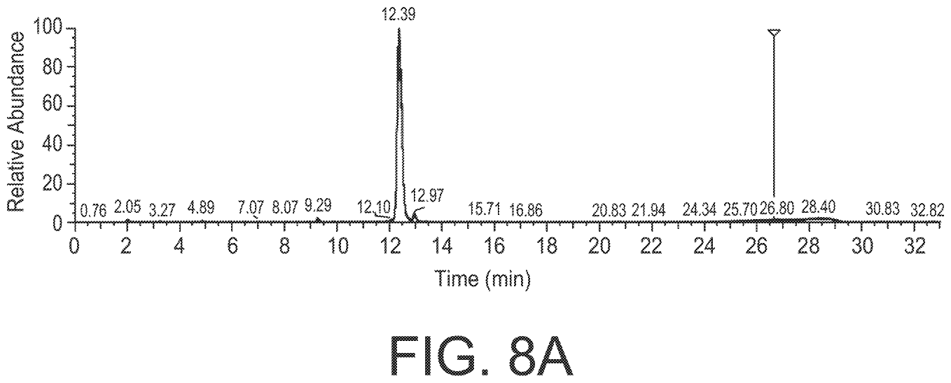

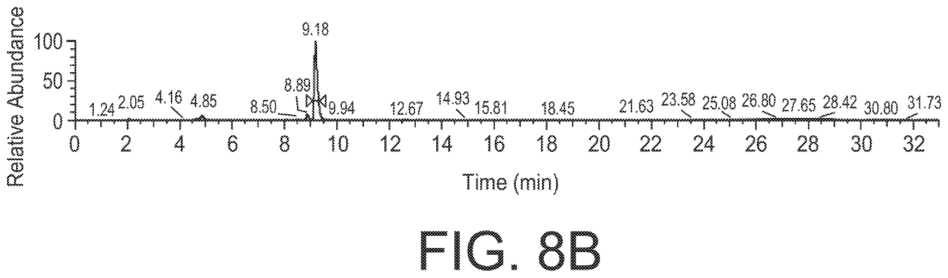

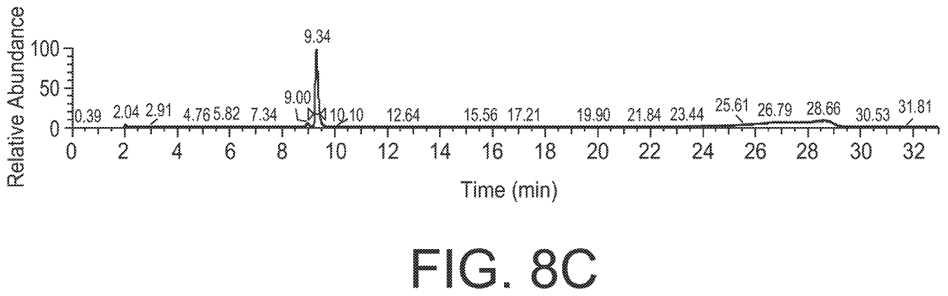

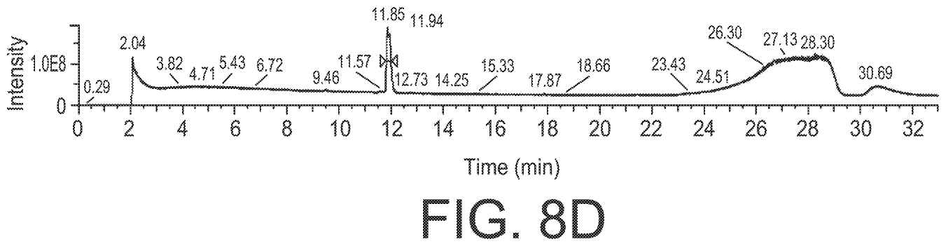

The oligonucleotide is a polyT of 20 thymines and a polyT of 11 thymines joined by a base uracil to obtain an asymmetrical cleavage product to enable a straight forward detection via length and mass on LC-MS. A sample of the solution including the 32-mer oligonucleotide was analyzed using LC-MS. The chromatogram generated from the LC-MS analysis of the solution including the 32-mer oligonucleotide is shown in FIG. 8A.

As shown in FIG. 8A, LC-MS results demonstrated a single peak of relatively high abundance at 12.39 minutes in the sample containing only the 32-mer oligonucleotide. Thus, the peak at 12.39 minutes corresponded to the intake 32-mer oligonucleotide.

This oligonucleotide was exposed to a UDG enzyme and a base AMA mixture (as described in Example 1) to excise the uracil base at position 21 from the 5′ end of the 32-mer (SEQ ID NO: 9), resulting in release of a polyT of 20 bases with a phosphate group on the 3′ end:

(SEQ ID NO: 10)
5′ TTTTTTTTTTTTTTTTTTTT-Phos 3′

and a polyT of 11 bases with a phosphate group on the 5′ end:

(SEQ ID NO: 11)
5′ Phos-TTTTTTTTTTT 3′.

The sample was purified in between each step to remove the reaction enzyme before adding the sample to LC-MS. The purification step removed the shorter cleavage product in the first step, and therefore only the polyT of 20 bases is retained in the remaining steps. This example is focused on demonstrating reuse reaction steps on this polyT oligonucleotide. The chromatogram generated from the LC-MS of the sample with the polyT of 20 bases is shown in FIG. 8B, demonstrating a single peak of relatively high abundance at 9.18 minutes, representing the polyT of 20 bases with a 3′ phosphate group.

The cleavage product with a 3′ phosphate group was then reacted with an Antarctic phosphatase enzyme to repair the 3′ end of the oligonucleotide, and the removal of the 3′ phosphate group was confirmed by LC-MS. The chromatogram generated from the LC-MS of the polyT of 20 bases after removal of the 3′ phosphate group is shown in FIG. 8C, demonstrating a single peak of relatively high abundance at 9.34 minutes, representing the polyT of 20 bases with no 3′ phosphate group.

The oligonucleotide with the repaired 3′ end was then reacted with a TdT enzyme mixed with nucleotide. This reaction led to the extension of the 3′ end of the oligonucleotide by a single base, as the nucleotide used is protected using the aminoxy group on its 3′ hydroxyl. The protected nucleotide is specifically used in this example to ensure a controlled single addition for a cleaner readout. The reaction product here is a polyT with 21 bases with aminoxy at the 3′ end and has the sequence:

(SEQ ID NO: 12)
5′ TTTTTTTTTTTTTTTTTTTTT-ONH2 3′.

The reaction product was analyzed by LC-MS. The chromatogram is shown in FIG. 8D, demonstrating a peak at around 11.85 minutes, representing the extended polyT oligonucleotide with 21 bases and a 3′ aminoxy protecting group.

### Example 9: On-Chip Demonstration of Reuse

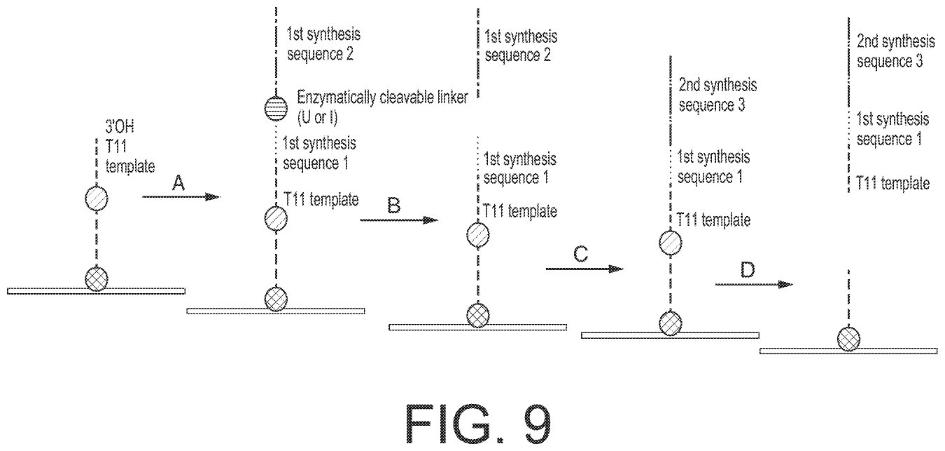

The present Example demonstrates reuse of a surface-tethered oligonucleotide on a chip. FIG. 9 is a schematic diagram showing the steps followed in this Example. The steps were designed for demonstration of reuse of the same starting oligonucleotide. The reuse can be repeated by incorporating the uridine base as many times as desired, given that the starting oligonucleotide is present for addition of bases by the TdT enzyme. In this Example, a photocleavable linker (PCL) was used to simplify the steps for demonstration purposes. In cases using an enzymatically cleavable linker, a base uridine nucleotide (dUTP) can be used. In other cases, nucleotides corresponding to Inosine (dITP) or modified versions of the bases can also be used. For example, a 3′ protected dUTP or dITP can be used to limit the addition of a cleavable linker to only a single base.

In the first step, the starting oligonucleotide was tethered to the surface of the chip. In this case, the chip surface was prepared with the following oligonucleotide sequence (in which PCL refers to a photocleavable linker) using phosphoramidite chemistry:

(SEQ ID NO: 13)
5′ TTTTTTT-PCL-TTTTTTTTTTT 3′.

In other examples, the tethered oligonucleotide could be attached to the surface using other attachment reactions, e.g., in which a purified oligonucleotide is covalently linked to functional groups on the surface, or in which an oligonucleotide is hybridized to another oligonucleotide that is attached to the surface. For example, the starting oligonucleotide can be prepared to have a modified 5′ terminating group that can react with a functional group that is added to the surface of the chip. In other cases, controlled addition of multiple cleavable bases or multiple cleavable bases along with other bases to generate sequence motifs that are highly efficiently cleaved by UDG can also be used. In those cases, the cleaving enzyme corresponding to the selected cleavable linker(s) will be used.

Step A, as shown in FIG. 9, was round 1 of enzymatic synthesis, in which sequence 1 was synthesized, and the cleavable linker that is the uridine base was incorporated into the sequence, followed by synthesis of sequence 2. Sequence 1 was synthesized to remain on the surface after sequence 2 is cleaved and extracted from the chip. Round 2 of synthesis, described below, resulted in sequence 3 and was performed on top of sequence 1 to demonstrate the second use of the same sites for synthesis. Specifically, the starting oligonucleotide was extended using TdT enzyme and respective nucleotides to extend the surface oligonucleotide further with the following oligonucleotide sequence:

(SEQ ID NO: 14)
5′ TTTTTTTTTT(U)ₓTTAATGGTAG 3′.

As the nucleotide corresponding to the base uridine was not protected, multiple uridine bases were added by the enzyme TdT, as represented by the (U)ₓ in SEQ ID NO: 14. The resulting oligonucleotide has the sequence:

(SEQ ID NO: 15)
5′ TTTTTTT-PCL-TTTTTTTTTTTTTTTTTTTTT(U)ₓTTAATGGTA
G 3′.

Step B, as shown in FIG. 9, was cleavage of sequence 2 from the surface using UDG and AMA reactions as described in previous Examples. The mass of the cleaved sequence 2 was then confirmed using LC-MS. Specifically, the surface was treated with the UDG enzyme, followed by AMA incubation, to perform cleavage of the sequence upstream of the uridine base. The resulting cleaved oligonucleotide of:

(SEQ ID NO: 16)
5′ Phos-TTAATGGTAG 3′

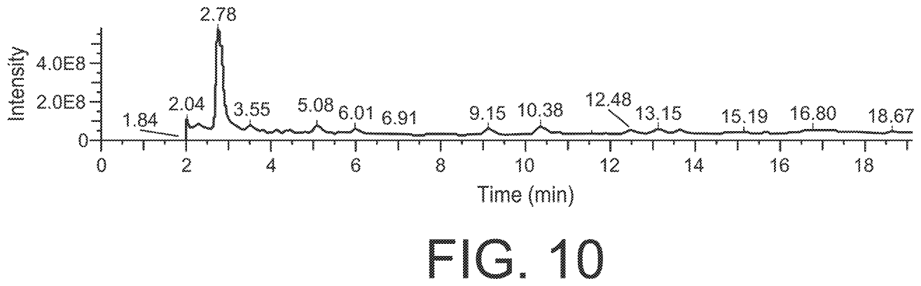

was collected, and the mass was confirmed by LC-MS. The chromatogram is shown in FIG. 10, demonstrating a peak at around 2.78 minutes, representing the cleaved oligonucleotide.

As shown in FIG. 9, sequence 1 was retained on the surface and had the sequence:

(SEQ ID NO: 17)
5′ TTTTTTT-PCL-TTTTTTTTTTTTTTTTTTTTT-Phos 3′.

The retained sequence had a 3′ phosphate group and was therefore treated with the phosphatase AnP to repair the synthesis site, i.e., to remove the 3′ phosphate left behind by UDG activity to give a free 3′ hydroxyl which can enable the next round of synthesis.

Step C, as shown in FIG. 9, was a second round of synthesis resulting in sequence 3. This step gave a combination of sequence 1 and sequence 3 on top of the starting oligonucleotide on the surface. Specifically, the surface was subjected to a second round of synthesis to add the sequence:
5′ GAT 3′.
   The final sequence after the second round of enzymatic synthesis was:

(SEQ ID NO: 18)
5′ TTTTTTT-PCL-TTTTTTTTTTTTTTTTTTTTTGAT 3′.

The second round of enzymatic synthesis containing part of the sequence synthesized in the first round of synthesis demonstrates the reuse concept.

In step D, as shown in FIG. 9, the combination of starting oligonucleotide, sequence 1, and sequence 3 was cleaved by exposing the PCL to UV light. The final sequence containing signatures of both synthesis rounds was then confirmed using LC-MS. The final sequence was:

(SEQ ID NO: 19)
5′ Phos-TTTTTTTTTTTTTTTTTTTTTGAT 3′.

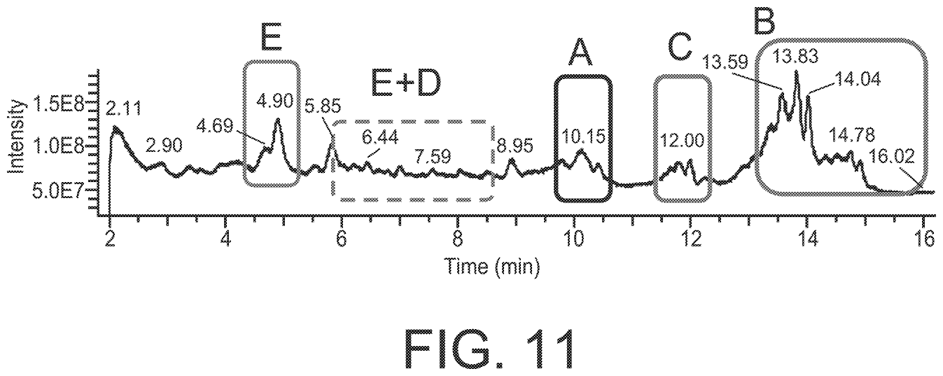

The chromatogram generated from the LC-MS is shown in FIG. 11. Since the reactions in the series of reactions described above were not fully optimized, there were several side products created in each step that were noted in the LC-MS data. The correct mass of the combined product of synthesis rounds 1 and 2 was noted at 10.15 minutes (marked with “A” in FIG. 11). FIG. 11 also includes various side products formed in the collection of steps taken towards reuse. The peaks marked with “B” represent side products from UDG reaction inefficiency. The peaks marked with “C” represent side products from uridine incorporation inefficiency. The peaks marked with “D” represent side products from phosphatase reaction inefficiency. The peaks marked with “E” represent side products from first synthesis cycle inefficiency. The individual reactions in this setup can be optimized to reduce side products and maximize the yield of final desired products. As seen in FIG. 11, the reaction efficiency of U cleavage when the oligonucleotide is tethered on the surface is lower than desired and can be improved by optimizing the reaction conditions. Improving this reaction may result in a higher percentage of sites available for reuse after cleavage.

The desired product was confirmed at high efficiency in the in-solution demonstration (Example 8) and to a detectable level in the tethered on-chip demonstration in the present Example, providing proof of concept of reuse as described herein.

All definitions, as defined and used herein, should be understood to control over dictionary definitions, definitions in documents incorporated by reference, and/or ordinary meanings of the defined terms.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A method for oligonucleotide synthesis, the method comprising:
providing a substrate including an oligonucleotide, the oligonucleotide including a first end bound to the substrate and a second end opposite the first end;
adding a cleavable base to the second end of the oligonucleotide;
enzymatically extending the oligonucleotide to generate a DNA molecule; and
cleaving the DNA molecule at the cleavable base.

2. The method of claim 1, wherein the DNA molecule is a first DNA molecule and the cleavable base is a first cleavable base, the method further comprising:
adding a second cleavable base to the second end of the oligonucleotide;
enzymatically extending the oligonucleotide to generate a second DNA molecule; and
cleaving the second DNA molecule at the second cleavable base.

3. The method of claim 1, wherein cleaving the DNA molecule at the cleavable base generates a 3′-phosphate group on the second end of the oligonucleotide, and the method further comprises:
enzymatically removing the 3′-phosphate group from the second end of the oligonucleotide.

4. The method of claim 1, wherein the substrate has a surface including a silicon surface, a silicon dioxide surface, a silicon nitride surface, a platinum surface, a gold surface, an indium tin oxide surface, a conductive surface, a non-conductive surface, or a composite surface.

5. The method of claim 4, wherein the surface is functionalized to enable binding of the oligonucleotide.

6. The method of claim 5, wherein the surface is functionalized with a silane coupling agent.

7. The method of claim 1, further comprising:
forming the oligonucleotide on the substrate via phosphoramidite chemistry.

8. The method of claim 1, wherein adding the cleavable base to the second end of the oligonucleotide comprises contacting the substrate with deoxyuridine triphosphate, terminal deoxynucleotidyl transferase, a coupling buffer, or a combination thereof.

9. The method of claim 1, wherein cleaving the DNA molecule comprises contacting the substrate to an enzyme capable of cleaving at the cleavable base.

10. The method of claim 9, wherein the cleavable base is a uracil base, and the enzyme comprises uracil DNA glycosylase.

11. The method of claim 10, wherein cleaving the DNA molecule further comprises contacting the substrate to a compound or enzyme capable of breaking a phosphodiester bond at an abasic site.

12. The method of claim 10, wherein the enzyme further comprises Endonuclease VIII or Endonuclease IV.

13. The method of claim 9, wherein the cleavable base is an inosine base, and the enzyme comprises Endonuclease V.

14. The method of claim 3, wherein enzymatically removing the 3′-phosphate group from the second end of the oligonucleotide comprises exposing the substrate to a 5′-phosphatase enzyme.

15. The method of claim 14, wherein the 5′-phosphatase enzyme comprises T4 polynucleotide kinase, Antarctic phosphatase, calf intestinal alkaline phosphatase, or a combination thereof.

16. A method for oligonucleotide synthesis, the method comprising:
providing an oligonucleotide comprising a first end and a second end;
adding a cleavable base to the second end of the oligonucleotide;
contacting the oligonucleotide to a substrate, wherein the contacting binds the first end of the oligonucleotide to the substrate;
enzymatically extending the oligonucleotide to generate a DNA molecule;
cleaving the DNA molecule at the cleavable base, thereby generating a 3′-phosphate group on the second end of the oligonucleotide; and
enzymatically removing the 3′-phosphate group from the second end of the oligonucleotide.

17. The method of claim 16, wherein the DNA molecule is a first DNA molecule and the cleavable base is a first cleavable base, the method further comprising;
adding a second cleavable base to the second end of the bound oligonucleotide;
enzymatically extending the oligonucleotide to generate a second DNA molecule; and
cleaving the second DNA molecule at the second cleavable base.

18. The method of claim 16, wherein cleaving the DNA molecule at the cleavable base generates a 3′-phosphate group on the second end of the oligonucleotide, and the method further comprises:
enzymatically removing the 3′-phosphate group from the second end of the oligonucleotide.

19. The method of claim 16, wherein the substrate comprises a silicon surface, a silicon dioxide surface, a silicon nitride surface, a platinum surface, a gold surface, an indium tin oxide surface, a conductive surface, a non-conductive surface, or a composite surface.

20. The method of claim 19, wherein the surface is functionalized to enable binding of the oligonucleotide.

21. The method of claim 20, wherein the surface is functionalized with a silane coupling agent.

22. The method of claim 16, further comprising forming the oligonucleotide on the substrate via phosphoramidite chemistry.

23. The method of claim 16, wherein adding the cleavable base to the second end of the oligonucleotide comprises contacting the substrate with at least one of deoxyuridine triphosphate, terminal deoxynucleotidyl transferase, or a coupling buffer.

24. The method of claim 16, wherein cleaving the DNA molecule comprises contacting the substrate to an enzyme capable of cleaving at the cleavable base.

25. The method of claim 24, wherein the cleavable base is a uracil nucleotide, and the enzyme comprises uracil DNA glycosylase.

26. The method of claim 25, wherein cleaving the DNA molecule further comprises contacting the substrate to a compound or enzyme capable of breaking a phosphodiester bond at an abasic site.

27. The method of claim 25, wherein the enzyme further comprises Endonuclease VIII or Endonuclease IV.

28. The method of claim 24, wherein the cleavable base is an inosine nucleoside, and the enzyme comprises Endonuclease V.

29. The method of claim 16, wherein enzymatically removing the 3′-phosphate group from the second end of the oligonucleotide comprises exposing the substrate to a 5′-phosphatase enzyme.

30. The method of claim 29, wherein the 5′-phosphatase enzyme comprises T4 polynucleotide kinase, Antarctic phosphatase, calf intestinal alkaline phosphatase, or a combination thereof.

31-40. (canceled)


SEQUENCE LISTING (19 sequences)

SEQ ID NO: 1 - DNA, 45 bp, synthetic construct
  source 1..45: mol_type = other DNA
  modified_base 15: mod_base = OTHER; note = uracil
ttttttttttttttttgcaatgcagtaccgagatcagtcagtgtc

SEQ ID NO: 2 - DNA, 48 bp, synthetic construct
  source 1..48: mol_type = other DNA
  modified_base 15: mod_base = OTHER; note = uracil
  modified_base 16: mod_base = OTHER; note = uracil
  modified_base 17: mod_base = OTHER; note = uracil
  modified_base 18: mod_base = OTHER; note = uracil
tttttttttttttttttttgcaatgcagtaccgagatcagtcagtgtc

SEQ ID NO: 3 - DNA, 11 bp, synthetic construct
  source 1..11: mol_type = other DNA
  modified_base 1: mod_base = OTHER; note = uracil
ttttttttttt

SEQ ID NO: 4 - DNA, 18 bp, synthetic construct
  source 1..18: mol_type = other DNA
  misc_feature 7: note = the thymine at position 7 is joined to the thymine at position 8 via a photocleavable linker
tttttttttttttttttt

SEQ ID NO: 5 - DNA, 11 bp, synthetic construct
  source 1..11: mol_type = other DNA
  misc_feature 1: note = 5' phosphate group
ttttttttttt

SEQ ID NO: 6 - DNA, 22 bp, synthetic construct
  source 1..22: mol_type = other DNA
  modified_base 12: mod_base = OTHER; note = uracil
  misc_feature 12: note = one or more additional uracil residues may be present between the uracil at position 12 and the thymine at position 13
tttttttttttttttttttttt

SEQ ID NO: 7 - DNA, 10 bp, synthetic construct
  source 1..10: mol_type = other DNA
tttttttttt

SEQ ID NO: 8 - DNA, 29 bp, synthetic construct
  source 1..29: mol_type = other DNA
  misc_feature 7: note = the thymine at position 7 is joined to the thymine at position 8 via a photocleavable linker
  modified_base 19: mod_base = OTHER; note = uracil
  misc_feature 19: note = one or more additional uracil residues may be present between the uracil at position 19 and the thymine at position 20
ttttttttttttttttttttttttttttt

SEQ ID NO: 9 - DNA, 32 bp, synthetic construct
  source 1..32: mol_type = other DNA
  modified_base 21: mod_base = OTHER; note = uracil
tttttttttttttttttttttttttttttttt

SEQ ID NO: 10 - DNA, 20 bp, synthetic construct
  source 1..20: mol_type = other DNA
  misc_feature 20: note = 3' phosphate group
tttttttttttttttttttt

SEQ ID NO: 11 - DNA, 11 bp, synthetic construct
  source 1..11: mol_type = other DNA
  misc_feature 1: note = 5' phosphate group
ttttttttttt

SEQ ID NO: 12 - DNA, 21 bp, synthetic construct
  source 1..21: mol_type = other DNA
  misc_feature 21: note = 3' aminoxy group
ttttttttttttttttttttt

SEQ ID NO: 13 - DNA, 18 bp, synthetic construct
  source 1..18: mol_type = other DNA
  misc_feature 7: note = the thymine at position 7 is joined to the thymine at position 8 via a photocleavable linker
tttttttttttttttttt

SEQ ID NO: 14 - DNA, 21 bp, synthetic construct
  source 1..21: mol_type = other DNA
  modified_base 11: mod_base = OTHER; note = uracil
  misc_feature 11: note = one or more additional uracil residues may be present between the uracil at position 11 and the thymine at position 12
tttttttttttttaatggtag

SEQ ID NO: 15 - DNA, 39 bp, synthetic construct
  source 1..39: mol_type = other DNA
  misc_feature 7: note = the thymine at position 7 is joined to the thymine at position 8 via a photocleavable linker
  modified_base 29: mod_base = OTHER; note = uracil
  misc_feature 29: note = one or more additional uracil residues may be present between the uracil at position 29 and the thymine at position 30
tttttttttttttttttttttttttttttttaatggtag

SEQ ID NO: 16 - DNA, 10 bp, synthetic construct
  source 1..10: mol_type = other DNA
  misc_feature 1: note = 5' phosphate group
ttaatggtag

SEQ ID NO: 17 - DNA, 28 bp, synthetic construct
  source 1..28: mol_type = other DNA
  misc_feature 7: note = the thymine at position 7 is joined to the thymine at position 8 via a photocleavable linker
  misc_feature 28: note = 3' phosphate group
tttttttttttttttttttttttttttt

SEQ ID NO: 18 - DNA, 31 bp, synthetic construct
  source 1..31: mol_type = other DNA
  misc_feature 7: note = the thymine at position 7 is joined to the thymine at position 8 via a photocleavable linker
ttttttttttttttttttttttttttttgat

SEQ ID NO: 19 - DNA, 24 bp, synthetic construct
  source 1..24: mol_type = other DNA
  misc_feature 1: note = 5' phosphate group
tttttttttttttttttttttgat